(19) United States
(12) Patent Application Publication
(10) Pub. No.: US 2015/0346177 A1
(43) Pub. Date: Dec. 3, 2015

(54) TEST STRIP AND OPTICAL ANALYSIS SYSTEM

(71) Applicant: TechIP International Limited, Larnaca (CY)

(72) Inventor: Eyal Dov Vardi, Bet Nir (IL)

(73) Assignee: TechIP International Limited, Larnaca (CY)

(21) Appl. No.: 14/287,411

(22) Filed: May 27, 2014

(51) Int. Cl. G01N 33/18

(52) U.S. Cl. CPC G01N 33/18

(57) ABSTRACT

An example test strip includes a liquid absorbing material, a reagent test portion, and an outer shell that encompasses the liquid absorbing material and the reagent test portion. The liquid absorbing material extends along the test strip and is designed to communicate liquid received through the outer shell to the reagent test portion. The outer shell is formed from a liquid impermeable material and includes a window area that allows an externally provided optical sensor to acquire an optical signal from the reagent test portion. The outer shell also includes a liquid transmission portion that is configured to allow liquid to flow into the area encompassed by the outer shell.

### CROSS-REFERENCE TO RELATED APPLICATIONS

The contents of U.S. application Ser. No. 13/742,089 (Attorney Docket No. 5871-8) are hereby incorporated by reference in its entirety.

### FIELD

The technology herein generally relates to test strips and systems using test strips in a diaper or other garment.

### BACKGROUND AND SUMMARY

Test strips, when exposed to certain bodily fluids, are useful tools that can give ordinary (or professional) people meaningful information on the current condition of the body. For example, a test strip that is impregnated with certain chemicals changes color when urine, blood, or some other body-related substance comes into contact with the strip—and some tests trips may simply react to wetness regardless of the type of liquid involved (or the presence of a substance in that liquid). Some test strips are designed with multiple test areas with each test area having a different chemical reagent for providing different types of information. For example, a single test strip (with multiple different test areas) may test for protein, hormone, glucose, and PH levels. To differentiate between tests, each test area on the test strip is designed to turn to a specific color should the concentration of a corresponding substance in question exceed a given amount.

Traditionally, tests of this type may have been carried out by placing the bodily fluid of the person into a container and submerging the test strip into the container. However, this type of laboratory environment may be relatively inefficient (or messy). For example, a person that is being tested for a urinary tract infection (UTI) may need to wait while the test is sent to a laboratory. This delay increases notification time and the costs associated with determining the health status of the patient. Thus, it will be appreciated that new and interesting techniques in this area are continually sought.

In certain example embodiments, a test strip may be provided with a reagent test area and a liquid absorbing material. The liquid absorbing material may extend along a length of the test strip and, in operation, may communicate liquid received at one location of the test strip to another location of the test strip where the reagent test area is disposed (e.g., through capillary action). An outer shell may encompass or be wrapped around the reagent test area and the liquid absorbing material. The outer shell is preferably at least one-way liquid impermeable at certain locations (e.g., made out of a liquid impermeable material, but perforated at some parts). The outer shell may be configured to prevent liquid from leaving the enclosed area once it has entered and thus encouraging liquid travel to a reagent test area. One portion of the outer shell opposite the reagent test area(s) may serve as a “window portion” that allows optical signal acquisition of the enclosed reagent test area from outside the test strip.

Another portion of the outer shell may include a liquid transmission portion that is configured to encourage and/or allow liquid to enter into the enclosed area and be absorbed into the liquid absorbing material. This liquid transmission portion of the outer shell, unlike the rest of the outer shell (e.g., which may be liquid impermeable), allows liquid to pass into the interior of the outer shell. Once absorbed by the liquid absorbing material, the liquid may be communicated “up” (or “down,” or “sideways,” etc) to the reagent test area(s). The enclosed and otherwise liquid impermeable nature of the outer shell may facilitate or assist in the communication of the liquid from one location or area of the test strip to another location or area (e.g., a reagent test area).

In certain example embodiments, a liquid analysis system is also provided. This example system includes a test strip, a housing, an optical sensor, and a processing circuit (e.g., a microcontroller, central processing unit, associated memory, input/output circuits, or the like). The test strip may include a pair of conductive layers or strips. The housing may be attachable to the test strip and to a garment and may include at least one surface with a plurality of conductive contact areas. The conductive contact areas may be configured to electrically communicate with the pair of conductive strip paths when the test strip is properly aligned. The optical sensor(s) may be carried by the housing and orientated towards the test area(s) of the test strip when the housing is attached to the garment. The processing circuit may be configured to use the pair of conductive strips and contact areas to perform an alignment verification process to ensure that the test strip (e.g., the test area) is properly aligned with the housing (e.g., the optical sensor). This alignment verification process may, for example, be based on determining whether or not the plurality of conductive contact areas are all (or mostly) in electrical communication with a pair of parallel conductive strips on the test strip.

### BRIEF DESCRIPTION OF THE DRAWINGS

These and other features and advantages will be better and more completely understood by referring to the following detailed description of exemplary non-limiting illustrative embodiments in conjunction with the drawings of which:

FIG. 1A is a perspective view showing a diaper with a test strip and an attached diaper tag located at the back of the diaper according to certain example embodiments;

FIG. 1B is a perspective view showing a diaper with a test strip and an attached diaper tag located at the front of the diaper according to certain example embodiments;

FIG. 1C is a side view of a person wearing a diaper with the test strip and diaper tag installed at the front position shown in FIG. 1B;

FIG. 2A and FIG. 2B are top views of example test strips according to certain example embodiments;

FIG. 2C is a cross-sectional side view of the test strip in FIG. 2A;

FIG. 3 is a perspective view of an example tag device used to acquire test strip data;

FIG. 4A is a side view of an example tag device;

FIG. 4B is a cross-sectional view of the example tag device in FIG. 4A;

FIG. 5 is a side view of an example tag device with an installed example test strip;

FIG. 6 is a block diagram of a tag device installed with an example test strip;

FIG. 7 is a flow chart of an example process of installing and using a tag device with a test strip according to certain example embodiments; and

FIG. 8 is a block diagram of an example computing system according to certain example embodiments.

### DETAILED DESCRIPTION

The following description is provided in relation to several example embodiments that may share common characteristics and/or features. It is to be understood that one or more features of any of the embodiments may be combinable with one or more features of other example embodiments. In addition, any single feature or combination of features in any of the embodiments may constitute an additional embodiment.

The example embodiments described herein may relate to test strips, diapers, and/or diaper tags for acquiring optical signals or images from a test strip (or areas of a test strip). In certain example embodiments, a diaper tag (including at least one optical sensor) communicates (e.g., wirelessly) with a remote computing resource where test results may be further processed.

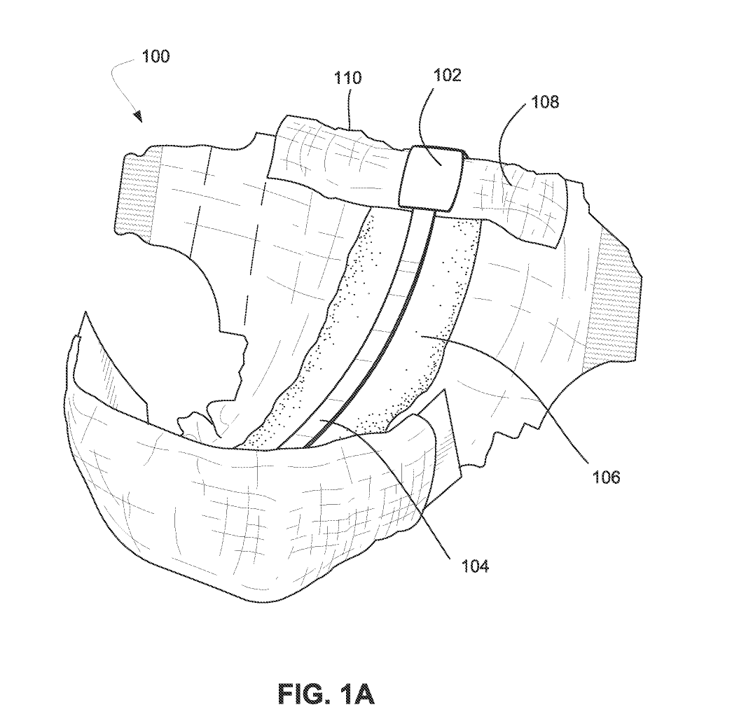

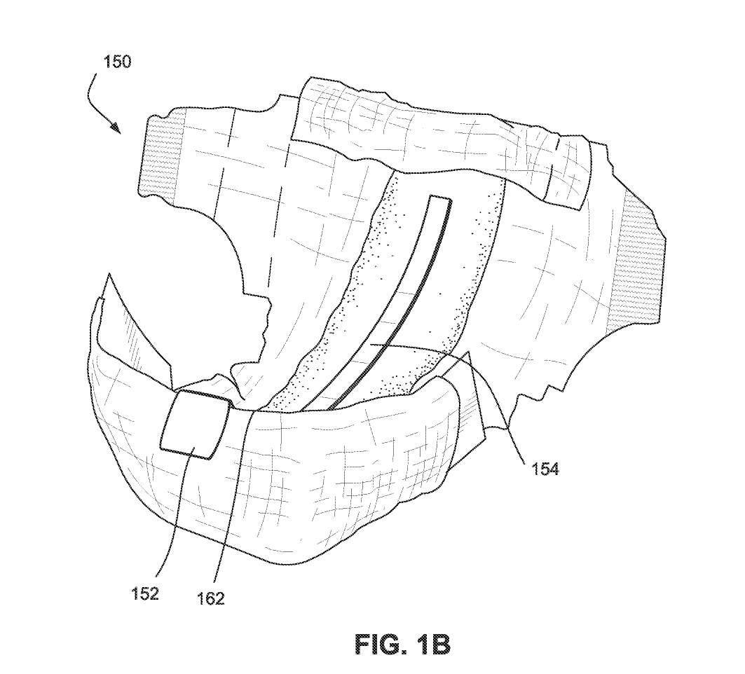

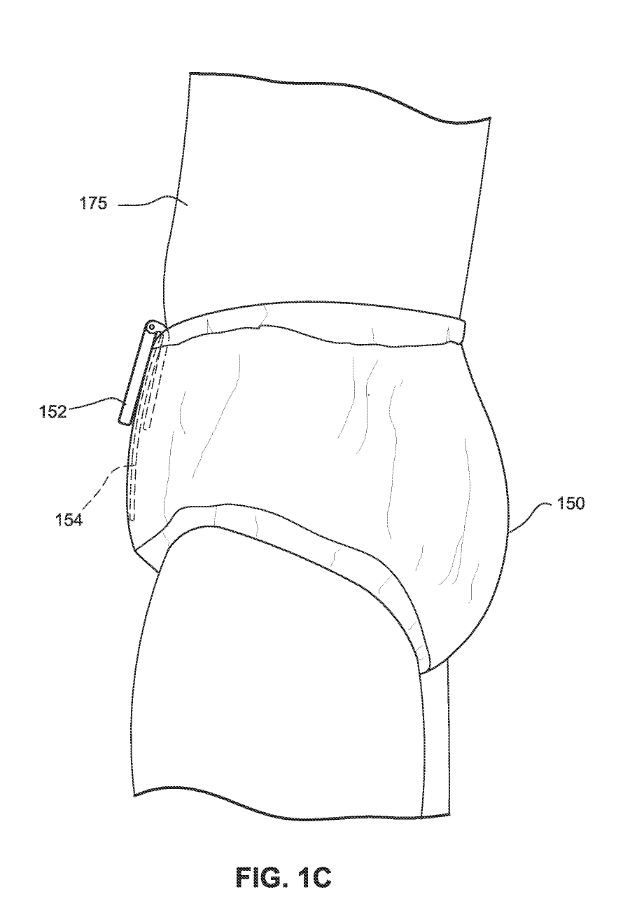

FIG. 1A is a perspective view showing a diaper with a test strip and a diaper tag according to certain example embodiments. Diaper 100 may be formed out of a cloth-like (e.g., cotton, microfiber, etc) material and/or other material (e.g., polymer based) that may be disposable. It will be appreciated that diapers may be differently sized for a comfortable fit depending on the wearer (e.g., babies, adults, etc).

A flexible test strip 104 is disposed inside of the diaper 100 on inner portion 106 and generally extends longitudinally along the middle of the diaper 100. A proximal end of test strip is positioned at or near the back end of the diaper 100 at rear edge 110 in FIG. 1A or the front end of the diaper 150 in FIG. 1B. The test strip 104 may be divided between a portion that is covered by the diaper tag 102 (an area of the test strip that includes at least one reagent test area) and an exposed portion. Generally, the portion of the test strip 104 that is exposed includes a liquid absorbing material (e.g., paper, tissue, etc) that communicates the liquid (e.g., by capillary action) to the covered reagent test area(s) of the test strip. Once the liquid reaches the test area(s), chemically impregnated test areas react to the liquid to thereby produce an optically sensible test result.

The re-usable diaper tag 102 attaches to the diaper at rear edge 110 in FIG. 1A and covers the proximal reagent test area of the test strip 104 and some of diaper reinforced area 108.

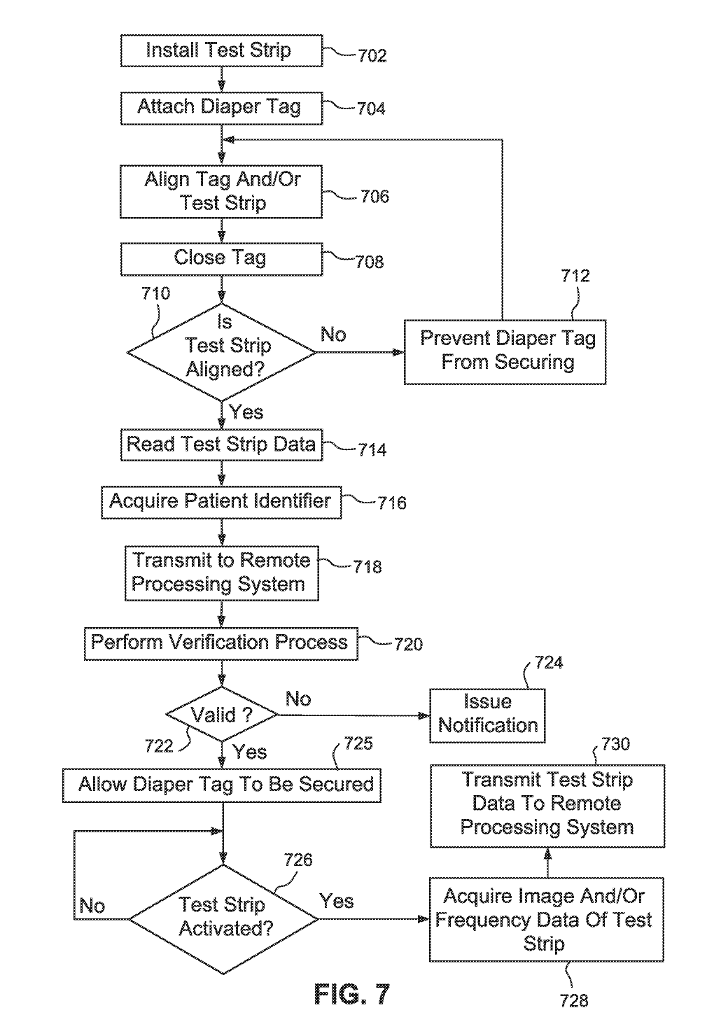

In certain example embodiments, so-called off-the-shelf test strips that may be bought at a typical pharmacy or the like may be adapted for use as a test strip with a diaper and/or associated diaper tag. However, preferably, the test strip(s) described herein may be proprietary custom made and used only with certain example proprietary diaper tags. In certain examples, diaper tags (e.g., device tags) may be configured to only operate with a particular proprietary test strip design. For example, a diaper tag may not initialize or function properly if a corresponding test strip is not a test strip recognized by the tag system as an authorized or valid test strip. Example techniques of verifying an installed test strip are described in connection with FIG. 7.

FIG. 1B shows an example embodiment of diaper 150 with test strip 154. Here, diaper tag 152 is located on a front portion of the diaper 150 (e.g., the diaper tag is designed to be located proximate to the stomach of a wearer of the diaper 150). Accordingly, the test strip 154 may be disposed on an inner surface of diaper 150 and extend from the “front” of the diaper where the test areas are located. Diaper tag 152 can then be secured at front edge 162 of diaper 150.

FIG. 1C is a side view of a person 175 wearing the diaper 150 of FIG. 1B. As can be seen, the test strip 154 is located on the front portion of the diaper 150 with the diaper tag being attached to the front edge of the diaper and over the test strip. The length of the test strip may vary depending on particular application needs. For example, the test strip may extend between the legs of the person wearing the diaper and be longer or shorter as required depending on diaper and/or patient size.

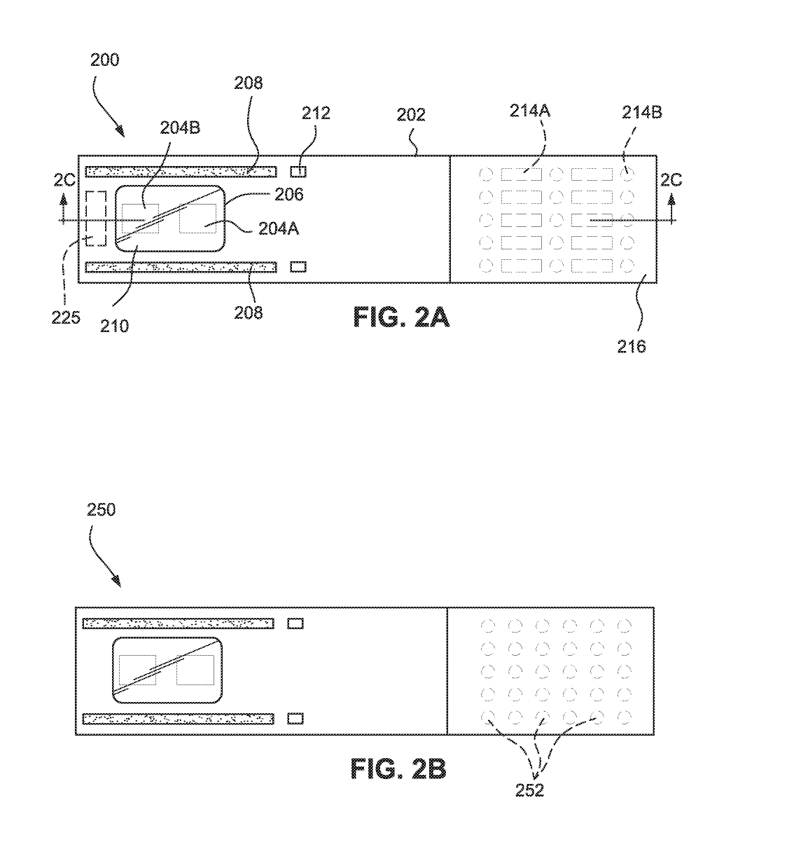

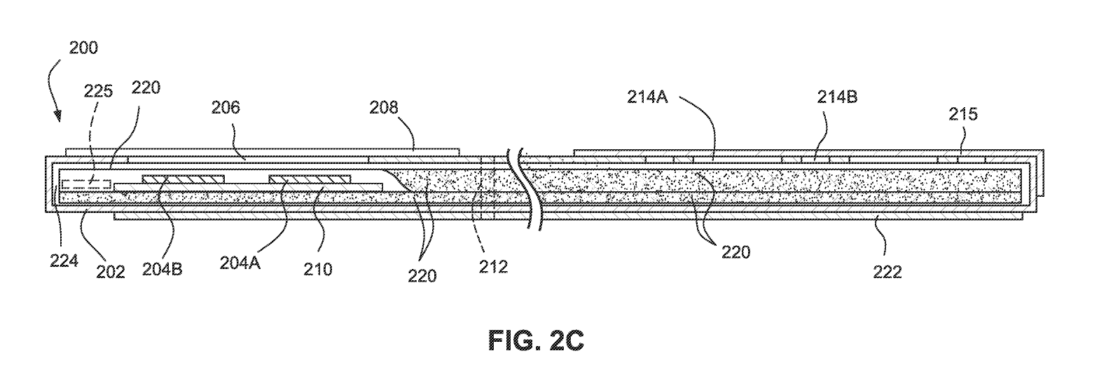

FIG. 2A and FIG. 2B are top views of example test strips according to certain example embodiments. FIG. 2C is a cross-sectional side view of the test strip in FIG. 2A.

Test strip 200 includes a flexible holder 202 that may be formed out of flexible nylon or other polymer. In certain example embodiments, the holder is constructed out of a flexible material such that the test strip 200 may be self-molded to different and changing surfaces—such as the inner surface of a diaper. In certain example embodiments, holder 202 is opaque for a majority of the external surface area. Making the holder 202 of the test strip 200 opaque can be advantageous as it may hide the liquid (e.g., urine) that is contained within the holder.

The holder 202 may be formed out of a material that does not allow liquid to pass there-through—e.g., the holder is liquid impermeable or substantially so. The liquid impermeability of the holder can be advantageous as it can prevent liquid outside of the test strip 200 from mixing with liquid inside the holder. Furthermore, the chemicals that comprise the reagent test areas can be prevented from coming into contact with the skin of a person. In other words, when reagent test areas come into contact with a liquid, some portion of the chemical reagents may transfer from the test area to the liquid. These chemicals could then be transferred away from the test areas and potentially contact the skin of a wearer, which may be disadvantageous. However, as the holder of a test strip may contain the liquid that comes into contact with the test areas, the reagents can be similarly contained and prevented (e.g., by providing a decreased likelihood of this occurring) from coming into contact with the skin of a user. Thus, liquid that enters into an area encompassed by the holder 202 may be trapped and not allowed to flow out of the holder while remaining free to travel within the holder by capillary action. As explained in greater detail below, certain portions of the holder may be designed to allow liquid to pass through the otherwise liquid impermeable shell of the holder and into an inner area—e.g., via a hole or aperture in the holder.

In certain examples, the size of holder (e.g., the test strip 200) is approximately 25 mm wide, 160 mm in length, and 3 mm in thickness. It will be appreciated, however, that other dimensions are possible depending on the particular application for the holder in connection with example test strips. For example, small, medium, and large sizes may be developed and used depending on the particular patient in question. Differently sized test strips may also be provided for male and female patients.

An interior area 224 formed by the holder 202 includes one or more reagent test areas. The example test strip in FIG. 2A shows two reagent test areas 204A and 204B, but the number of reagent test areas included with a test strip may vary. For example, a test strip may include 5 or 10 different reagent testing areas. The particular tests associated with each test area may be tests that are approved by a suitable government agency (e.g., the U.S. Food and Drug Administration) or other tests that are developed based on particular application needs.

In certain example embodiments, reagent test areas are designed to test for nitrites that may be produced as a result of a urinary tract infection in a patient. Alternatively, or in addition, the reagent test areas may test for leukocytes (e.g., white blood cells). It will be appreciated that other test types may be used in conjunction with the example embodiments described herein.

Reagent test areas 204A and 204B are typically disposed on a liquid permeable base surface 210 and located near one (lengthwise) end of the test strip 200. In certain examples, the placement of the reagent test areas 204A and 204B may serve as a basis for aligning reagent test areas 204A and 204B with window 206 to allow for optical sensors or the like (described in greater detail below) to acquire information on the state of reagent test areas 204A and 204B.

In certain example embodiments, window 206 is formed out of the same general material of the rest of holder 202. For example, a majority of the holder 202 may be formed out of opaque nylon while the window 206 portion may be formed out of clear, translucent, or transparent nylon (or another transparent, translucent, or clear polymer). In certain example embodiments, the window may be a portion of the holder that is removed—e.g., an aperture in the holder may be provided to allow for visual acquisition of the reagent test areas. Generally speaking, the “window” area of the holder may be designed to allow optical acquisition of a color (or even a pattern or an image) of a particular reagent test area. In certain example embodiments, all or a majority of the holder may be constructed out of the same material used for the window 206. In other words, the holder may be entirely made out of a clear, translucent, or transparent material.

A passive RFID device 225 is provided within test strip 200 and may contain information on the test types supported by a particular test strip embodiment. In certain examples, the RFID device may include an authentication code or similar data used to verify that an installed test strip is compatible (or authorized) with that particular device tag. The passive RFID device 225 captures the electromagnetic energy produced by an externally provided exciter and utilizes that energy to send back data. For example, an RF exciter and RF reader may be disposed to be in RF communication with an example tag device.

On opposing sides of window 206 parallel conductive strips 208 formed out of conductive ink are printed or otherwise disposed on holder 202. As explained in greater detail below, the conductive strips may be used to verify or confirm alignment, position, and/or existence of the test strip with respect to an example device tag. In certain examples, the size of the conductive strips may be approximately 4 mm×20 mm, although other lengths and widths are possible.

In certain examples, the length of the conductive strips and/or the separation distance between conductive strips 208 may be designed to match distance(s) between conductive contact elements of a device tag. For example, if the length of a conductive strip is about 20 mm then the distance between two conductive contact elements on a device tag may be about 20 mm (or slightly less). By having the length of the conductive strips approximately correspond to the distance between the conductive contact elements on the device tag, a system (e.g., a local or remote processor—perhaps part of RFID chip 225) may determine that the device tag and test strip are properly aligned with one another (e.g., by testing to see which conductive contact elements are electrically shorted together by contact with a mated conductive strip). In certain examples, one or more “inner” conductive contact elements may be included between two “outer” conductive elements of the device tag. For example, five conductive contact elements may be disposed on a device tag at increments of about 5 mm with a “distance” between the two outer conductive contact elements at about 20 mm. Accordingly, the respective conductive strips may each contact 5 respectively corresponding conductive contact elements that are disposed on the device tag to signify proper alignment between the device tag 102 and the test strip 200.

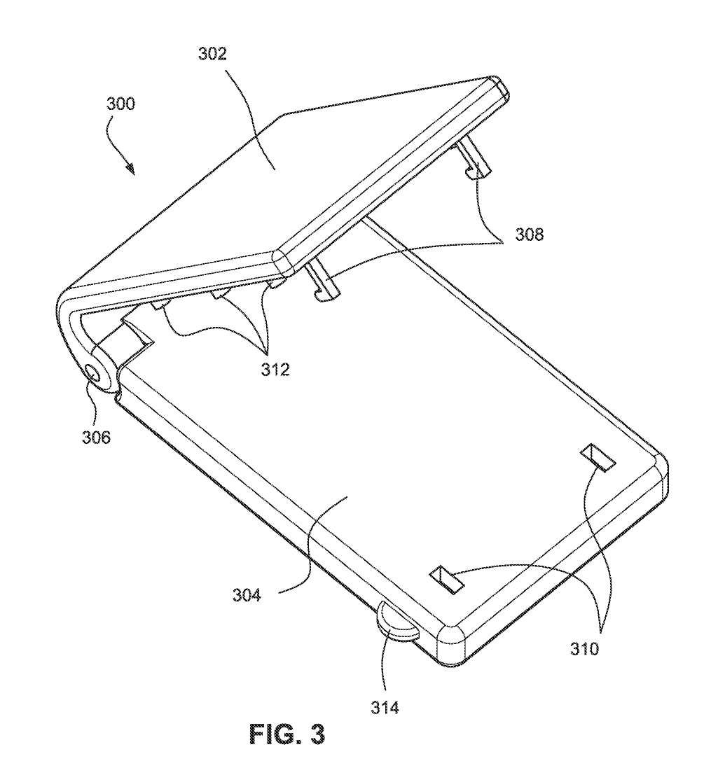

While FIG. 2A shows a straight line for the conductive strips 208, other patterns of conductive strip(s) or layer(s) (and matching tag contact elements) may be used. For example, a circle or zigzag pattern may be used to verify alignment, position, and/or existence of the test strip. In such situations, the arrangements of the conductive contact elements on the device tag should be adjusted to match the expected arrangement of the conductive strip on the test strip. For example, the conductive contact elements may be provided in a circular pattern on a device tag to match a corresponding circular conductive strip on a test strip. In certain examples, the width of the conductive layer on the test strip may also be designed to match the size of the conductive contact elements of a device tag. For example, the width of conductive strips 208 in FIG. 2A may match the width (or diameter) of conductive contact protrusions 312 from FIG. 3 that extend from the device tag 300.

Test strip 200 includes a pair of apertures 212 that extend through the test strip to accept attachment prongs from a diaper tag. The tag attachment apertures thus may allow the test strip to be secured to an example diaper tag. In certain examples, the apertures are formed after the test strip 200 has been assembled by punching through the material of the test strips (e.g., by using a hole punch). Accordingly, the attachment apertures may be formed through the holder and other layers of the test strip (e.g., the liquid absorbing material, the soft material layer, etc). In certain examples, apertures 212 are sealed to prevent liquid from entering the area (e.g., interior 224 in FIG. 2C) enclosed by the holder 202. In other words, despite apertures being formed through the test strip, the apertures preferably do not provide a path for the liquid to enter and/or exit to/from the area enclosed by the holder.

Soft material layer 216 may be layered on top of a portion of the holder 202. In certain examples, the soft material layer 216 is soft tissue paper and is designed to come into contact with the skin of a person that is using the test strip. Generally speaking the soft material layer is added for comfort of the person using the test strip. Soft material layer 216 is layered at a distal end of the strip that is opposite the location of the testing areas 204A and 204B.

FIG. 2A shows an example arrangement of holes with alternating small 214B and large 214A holes arranged within holder 202 near its distal end. FIG. 2B shows another example embodiment with multiple holes 252 that all have the same general geometry.

In certain example embodiments, a one-way liquid permeable membrane may be used in combination with at least one aperture provided in the holder. Such a membrane may allow liquid into the holder, but prevent it from easily leaving the interior of the holder. Such a membrane may be integrally formed with the remainder of the holder—e.g., via stitching or the like. The test strip may thus operate by allowing liquid (e.g., urine) to pass through a soft material layer, through apertures in the holder, and into the interior of the holder.

Turning now to FIG. 2C, which is a cross-section view of FIG. 2A, the interior 224 of holder 202 includes absorbent material 220 that transfers liquid (e.g., by capillary action) entering holder 202 at apertures 214A and 214B to reagent test areas 204A and 204B. As shown in FIG. 2C, holder 202 is a shell that encompasses absorbent material 220 and other components of test strip 200. The absorbent material may be made of the same material that diapers are made of—e.g., a cloth (cotton, hemp, etc) or synthetic substance. In certain examples, the absorbent material may be made of paper, such as, for example, a paper towel or tissue paper.

The transmission of liquid from one distal end of the test strip (e.g., the portion under the apertures) to the other proximal end (e.g., where the test areas are located) can be accomplished by the physical phenomena known as capillary action. This allows the liquid to “flow” with or against external forces (e.g., a force due to gravity). The absorbent material 220 extends from one end of the test strip towards the other and encompasses the reagent test areas 204A and 204B. In certain examples, a thin layer of absorbent material 220 (e.g., thin enough to still allow the test areas to be externally viewed through “window” 206 so as to discern a color of a particular test area) covers reagent test areas 204A and 204B. The thin layer of absorbent material 220 may facilitate delivery of liquid such that all (or most) of the test area has liquid applied thereto. Absorbent material 220 may be made of a material that becomes transparent (or substantially so) when liquid is absorbed. Thus, when the thin layer is dry it may optically occlude the test areas. However, when liquid is absorbed at the thin layer it may become transparent to allow optical signal acquisition from the test areas.

Base surface 210, upon which reagent test areas 204A and 204B are disposed, is in contact with absorbent material 220. The proximity of absorbent material 220 to reagent test areas 204A and 204B assists in providing the liquid to reagent test areas 204A and 204B from opposite end of test strip 200.

Holder 202 may also include an external adhesive strip 222 that can be used to adhesively attach the test strip 200 into or onto a diaper or the like (e.g., shorts, pants, or other garment). Adhesive strip 222 is bonded to holder 202 on one side and may include a protective removable layer that, when removed, exposes an opposing adhesive side for bonding adhesive strip 222 (and thus test strip 200) to a diaper or other garment.

In certain example embodiments, the test strip can be inserted or integrated into an existing garment. For example, a test strip may be stitched into the garment or inserted into a pre-configured holding area of the garment.

In certain example embodiments, a tag device is provided for analyzing and/or acquiring information related to reagent testing areas on an example test strip—e.g., the test strip 200 as described above. In certain example embodiments, a tag device is designed and configured to attach to a diaper or other garment worn by a person.

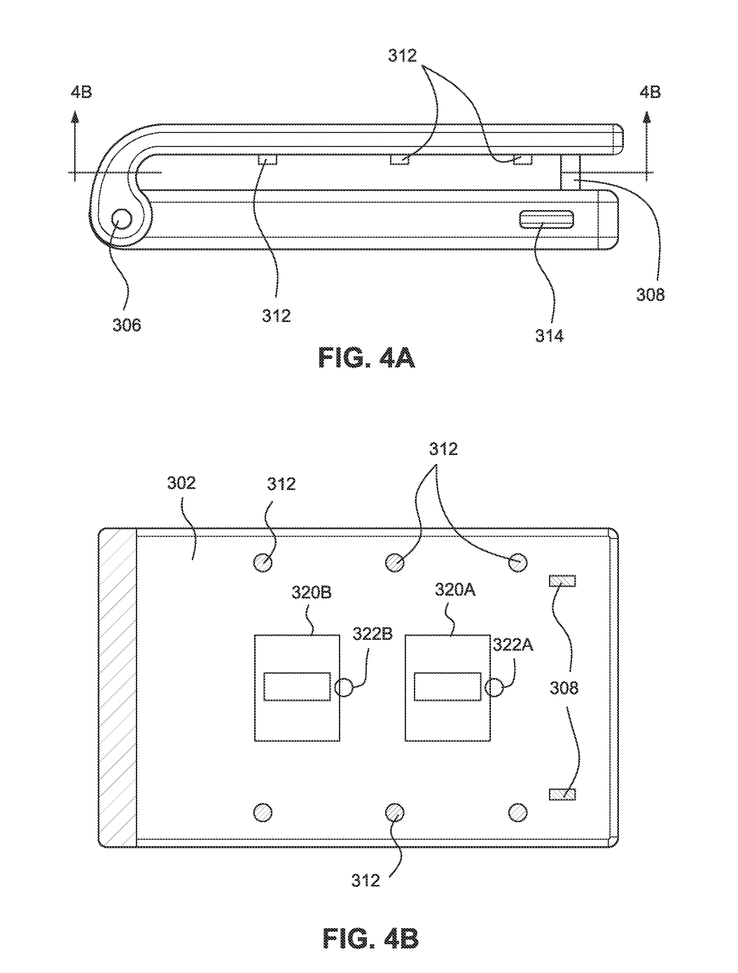

FIG. 3 is a perspective view of an example tag device used to acquire test strip data from example test strips. FIG. 4A is a side view of the tag device of FIG. 3 and FIG. 4B is a cross-sectional view of FIG. 4A.

Tag device 300 includes an upper portion 302 rotatably coupled to lower portion 304 by hinge 306. The view in FIG. 3 shows tag device 300 in an open position. The hinge 306 may include a spring or other mechanism that provides a biasing force to force the upper and lower portions away from one another.

Latch prongs 308 are configured to be accepted into cavities 310 such that upon insertion of latch prong 308 into cavities 310 the tag device 300 may be secured.

Once secured, tag device 300 can be released by pressing or otherwise triggering release button 314 that is disposed on a side of the lower portion 304 of tag device 300. In certain examples, un-securing the prongs from the cavities may cause the tag device to “spring” open—e.g., due to a biasing spring force provided by a hinge spring. Alternatively, or in addition, a person may pry the two portions of the tag device away from each other to open the tag device upon releasing the latch prongs from their respective latch cavities.

Tag device 300 includes conductive contact protrusions 312 on an inner surface of upper portion 302 of the tag device 300. The conductive protrusions 312 are designed to contact (e.g., firmly) with conductive strips or other conductive portions of an example test strip that is engaged with the tag device.

In certain example embodiments, conductive contact protrusions may be provided on the lower portion of the tag device instead of the upper portion. In certain examples, conductive elements of the test strip are disposed to contact conductive contact protrusions placed onto the lower portion of the tag device (e.g., a place “below” the reagent test areas or window of a tag device). In certain examples, conductive contact protrusions may be provided on both the upper and lower portions of the tag device and designed to contact corresponding conductive elements of a test strip.

Referring to FIG. 4B, the inner surface of upper portion 302 has optical sensors 320A and 320B disposed thereon. The optical sensors are designed and positioned on the tag device to “view” test areas of a test strip engaged with the tag device. Illumination of the test areas (e.g., to facilitate acquisition of optical information or an image) of a test strip is provided by small LED light sources 322A and 322B. As described in U.S. application Ser. No. 13/742,089, the contents of which are hereby incorporated by reference, the light sources may be selectively activated in accordance with acquisition of optical signals from test areas of a test strip.

In certain example embodiments, the optical sensor that is used may be a color light-to-frequency converter. An example optical sensor is the TCS3200 manufactured by Texas Advanced Optoelectronic Solutions® (TAOS). It will be appreciated that other types of cameras, scanners, images, optical sensors, or the like may be used to acquire optical signals or data related to the state of a testing area on a test strip. Such imagers may include color, grayscale, or black and white imagers.

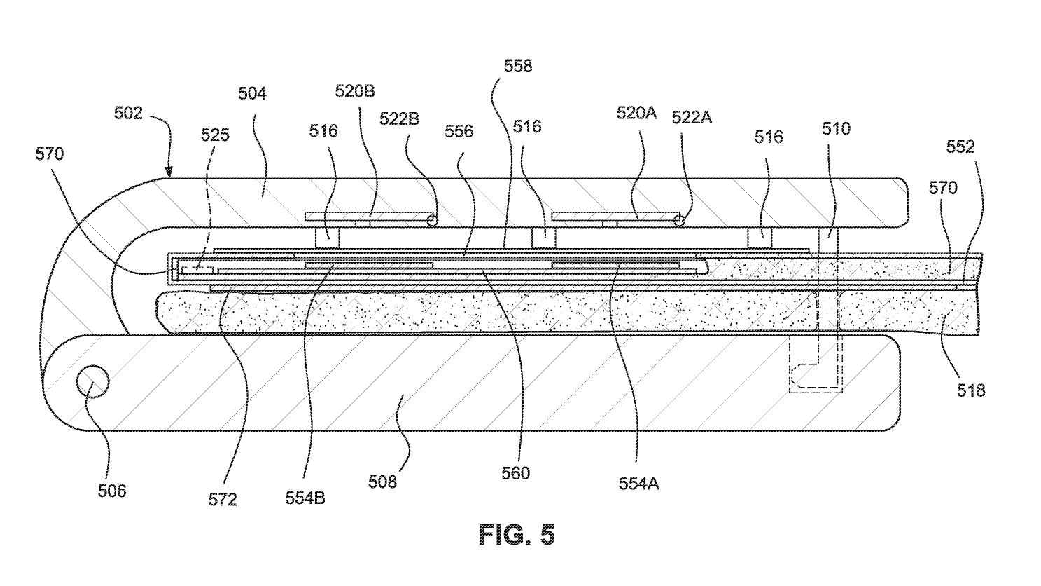

FIG. 5 is a side view of an example tag device 502 with an installed example test strip. Tag device 502 includes an upper portion 504 rotatably coupled to lower portion 508 by hinge 506.

Optical sensors 520A and 520B are disposed on/in upper portion 504 of the tag device. For example, a part of the optical sensors may be disposed within the structural body of the tag device while another portion may extend from the body of the upper portion of the tag device.

In operation, a tag device 502 is designed to fit around an edge of a diaper (or other garment) 518 that has a test strip attached thereto.

The test strip shown in FIG. 5 includes a holder 552 (e.g., formed out of nylon) that is affixed to diaper 518 by adhesive strip 572. An inner area of the holder includes absorbent material 570 used for communicating liquid to test areas 554A and 554B that are disposed on neutral base material 560 where liquid is received into the inner area.

Viewable window 556 is provided in holder 552 to allow optical sensors 520A and 520B to respectively acquire optical data (e.g., reflected light frequency data) relating to test areas 554A and 554B. LED light sources 522A and 552 may provide illumination for the test areas when obtaining such optical data. A passive RFID chip 525 is preferably deposed within holder 552 and may also be in circuit connection with conductive strips 558.

Tag device 502 is secured to diaper 518 and the corresponding test strip by prong(s) 510. Specifically, when tag device 502 is closed, prong(s) 510 extend(s) through mated aperture in the test strip and diaper into mated latch cavities provided in lower portion 508 of tag device 502. In certain examples, the prongs are designed to punch through a diaper.

In certain examples, other techniques for securing the test strip (and the diaper) to the tag device may be used. For example, instead of puncturing the diaper, or test strip, prongs may clamp over the test strip and diaper. In such instances, there may be no need for an aperture in the test strip and/or diaper.

In certain examples, an aperture is provided through the test strip but not the diaper. In other words, the latching prongs extend through the test strip and clamp to the diaper. The tag device may be held in place by a spring force or other securing technique (e.g., the hinge may be locked to a given position that secures the tag to the diaper and test strip).

The example test strip shown in FIG. 5 includes a conductive strip layer 558 provided on an outside surface of the holder 552. The conductive strip layer 558 is designed to conductively couple to conductive contact protrusions 516 that extend from an inner surface of upper portion 506. As explained below, the connection of the plural conductive contact protrusions to a conductive strip layer of the test strip may be used to test and ensure that the test strip is properly positioned and/or aligned with respect to the tag device (e.g., so that optical information of the test areas may be successfully obtained by optical sensors of the tag device).

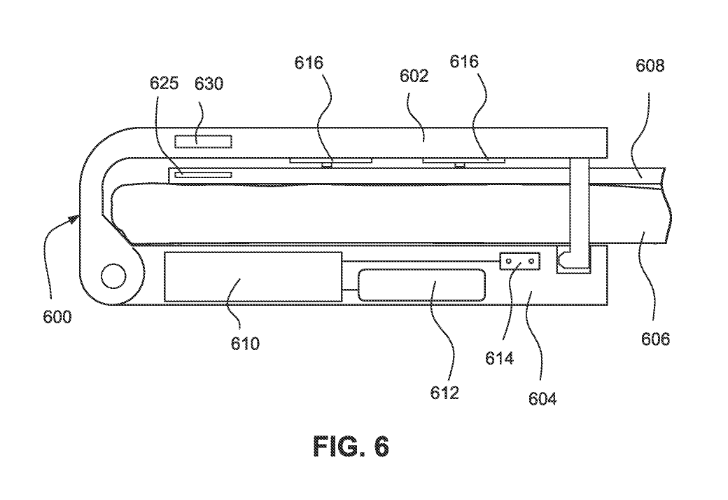

FIG. 6 is a block diagram of a tag device installed with an example test strip. Tag device 600 includes an upper portion 602 and a lower portion 604. The upper portion 602 includes a pair of optical sensors 616 that are for acquiring optical data of test strip 608 that is affixed to diaper 606. It will be appreciated that the number of optical sensors on the tag device may be adjusted based on specific application needs. For example, one optical sensor may be provided (e.g., if there is one test area within a test strip). Alternatively, more than one optical sensor imager may be provided in the tag device (e.g., if there is more than one possible test area in a test strip). If an array of test areas (either multi-dimensional or linear) is used then a similarly configured array of optical sensors may be provided in the device tag. In certain examples, an image can be acquired (e.g., via an imager or a camera) of the test areas and analyzed to detect the current state of each test area within the captured image.

Upper portion 602 of tag device 600 also preferably includes an RFID reader 630 configured to send electromagnetic energy in order to excite and communicate with passive RFID tag 625 provided in test strip 608.

The lower portion 604 preferably includes a printed circuit board (PCB) 610 coupled to battery 612. The PCB 610 may include an integrated circuit (IC) or microprocessor, one or more memory devices for storing test data, transceivers for sending and/or receiving data and commands by radio frequency (RF) communication channels, and other components. In certain examples, a processor is part of an RFID chip that is provided on the PCB and is used to communicate with external computing resources via RF signals. In certain examples, the PCB is or includes a system-on-a-chip (SoC) that provides signal and data processing and signal and transmission functionality.

PCB 610 is coupled to the optical sensors 616 and/or RFID reader 630 (e.g., via flexible connections that cooperate with the hinged connection) to allow for processing and/or routing of acquired optical data—e.g., as described in U.S. application Ser. No. 13/742,089, the contents of which are hereby incorporated by reference. In certain examples, the RFID reader may be provided on a lower portion of the tag device and communicate with passive RFID tag 625.

A recharging connector 614 is used for recharging battery 612 from an external source. In certain examples, the charging may be accomplished through induction and a coupling conductor plate may be hidden within the body of tag device 600. In other examples, a charging connection port is externally provided and configured to accept a connection from an external power source.

FIG. 7 is a flow chart of an example process of installing and using a tag device with a test strip according to certain example embodiments. In step 702, a test strip is installed or otherwise positioned within a diaper and in step 704 a diaper tag is fixed to the diaper to cover the testing area of the test strip.

In step 706, as part of the installation process of the test strip and the diaper tag, the tag and the test strip are aligned with respect to each other by a technician, nurse, or other person. This may involve moving the test strip, the diaper tag, or both.

In step 708, the diaper tag is closed about the disposed test strip. For example, a nurse or other person may engage attachment prongs of a tag device with corresponding receiving cavities.

In step 710, a computerized process is triggered to determine if the diaper tag is properly aligned with respect to the test strip. In certain examples, the trigger for this process occurs when attachment prongs come into contact with receiving cavities on the device tag (e.g., when an electrical circuit is completed via the latching prongs and the latching cavities). In certain example embodiments, this process is executed on components (e.g., a processor) of the diaper tag. In certain example embodiments, the processing may be performed on remotely provided computing resources that are wirelessly coupled to the tag device. In other words, the diaper tag may send (e.g., via a transceiver) sensed data about the placement of the diaper tag, the test strip, or a combination thereof to an external computing resource. In response, the external computing resource may provide a result that “answers” whether the test strip is properly aligned or not.

In certain example embodiments, the alignment process may include determining whether all (or some percentage) of the conductive contact protrusions have come into contact with one or more conductive strips of a tests strip. This process may include testing pairs of conductive contact protrusions to measure a resistance value between the respective conductive contact protrusions. If there is little or no resistance then it may be inferred that the conductive strip has electrically coupled the two contact protrusions.

For example, referring back to FIG. 2A and FIG. 4B, a processor or the like may be configured (e.g., via software, firmware, specialized hardware, etc) to determine whether or not (and which) conductive contact protrusions 312 are in contact with conductive layer 208. The determination may be accomplished by testing if a circuit is formed between the protrusions and the conductive layer (and possibly including components of the PCB).

With such techniques, an automated or programmatic process may determine that all of the contact protrusions are conductively coupled with a corresponding conductive layer of the test strip. In such a case (e.g., due to the dimensioning of the contact protrusions in relation to the conductive layer), the process may determine that the test strip and the tag device are properly aligned to each other. Specifically, if the two parallel lines formed by the conductive layer on the test strip (e.g., in FIG. 2A) contact all six of the contact protrusions (e.g., in FIG. 4B) the process may determine that the test strip is properly positioned with respect to the diaper tag.

In certain examples, less or more contact protrusions can be used on the device tag—e.g., 4 instead of 6 contact protrusions may be used. Other techniques of testing the alignment based on testing conductive connection(s) between the device tag and the test strip can also be used to facilitate alignment testing and verification.

In any event, a determination as to the alignment of the test strip with respect to the tag device is provided in step 710. If the process determines that the tag and test strip are not properly aligned, the tag may be prevented from securing in step 712. In certain example embodiments, components on the PCB of the device tag control engagement of the prongs with the cavities of the device tag such that if the alignment test fails the prongs will not be able to be securely engaged into the cavities. Alternatively, or in addition, a hinge that connects the upper and lower portions of a device tag is not locked into place (or is unlocked) if the alignment verification process does not succeed. In certain example embodiments, the prongs or other device tag components are controlled by an electro-mechanical lock or the like. In certain examples, the prevention aspect includes providing an audible beep or flashing light to notify a user that the device tag is not properly aligned with respect to the test strip. After disabling, preventing, or notifying the user can then attempt to re-align the device tag to test strip in step 706 again.

In step 714, data from a passive RFID tag located on or within the test strip is read (and excited) by a corresponding RFID reader. In certain examples, the RFID reader is a component of the diaper tag. Alternatively, or in addition, an RFID reader is provided with a wristband or a device carried by a staff member (e.g., a nurse). The RFID reader acquires data stored by with the RFID tag of the test strip. This data can include the type of test that is provided by the test strip and/or other information (e.g., an authentication code).

In step 716, an identifier for the patient that is going to be using the test strip is acquired. In certain examples, the identifier is stored with the diaper tag. For example, when a nurse or the like registers and/or initializes the diaper tag, an identifier of the patient may be assigned to that particular diaper tag (e.g., as preferably stored in on-board flash memory or the like).

In certain examples, the diaper tag may communicate with a band worn by a patient. Example bands are described in U.S. application Ser. No. 14/267,817 and U.S. Publication No. 2013/0182382, the entire contents of each being hereby incorporated by reference. For example, a diaper tag may wirelessly communicate with a band worn by a user to acquire a patient identifier that is stored within the band.

In step 718, identification information related to the type of test strip (e.g., a testID) and patient information (e.g., a patientID) may then be communicated (e.g., via wireless communication) to a remote processing system.

In step 720, the remote processing system can use this acquired information to determine if the particular test associated with the testID is a type of test that has been prescribed for that patient (patientID). In certain examples, an electronic medical records database can be consulted (e.g., programmatically accessed) to determine and/or verify the nature of the test that is currently being deployed to a patient. The result of this determination may be communicated back to the diaper tag (or some other destination).

In step 722, the diaper tag may receive the communication from the central system that indicates whether the test is valid for a particular patient.

If the test is not valid, then in step 724 the diaper tag may issue a notification (locally and/or to a remote computing system). In certain examples, if the test is found to be invalid then the remote processing system may send a communication to a staff member with a brief description of the problem (e.g., that patient X has been issued the incorrect test strip for Z). In certain examples, the notification may be a beep or the like that is issued by the diaper tag. In certain examples, as described above, the diaper tag may be prevented from properly securing to the diaper.

If the verification process is successful, then in step 725, the diaper tag is secured. In certain example embodiments, an electromechanical lock may be used to secure prongs in latch cavities of the device tag. Alternatively, or in addition, a sound or light may be provided that indicates that the device tag has been secured. In certain example embodiments, initialization routines on the diaper tag may be triggered. For example, a first image of the testing areas may be obtained using the imager or optical sensor of the diaper tag. This may be carried out to provide further verification that the diaper tag is ready for operation.

Once activated, the diaper tag may enter a loop that continually checks for activation of the test strip in steps 726. The technique used for detecting activation of a test strip may be one of those described in U.S. application Ser. No. 13/742,089, the contents of which are hereby incorporated by reference. For example, the diaper tag may detect the presence of an electrical short circuit between the conductive layers located on the test strip and/or the presence of wetness within view of tag device imaging sensors.

In certain examples, the diaper tag may continually acquire images, image data, or color data of the testing areas at predetermined (or otherwise programmatically set intervals). If there is no change in the state of the test strip the diaper tag may simply discard the acquired data and continue checking for activation of the test strip. In certain examples, the optical sensor(s) may acquire data at a rate of once every 60 seconds. In certain example embodiments, the diaper tag may continually check if a reagent test area is wet with any liquid. In certain examples, the reagent test areas may change color and/or shape based on the presence of a liquid in the test strip (even if the test of the test area is negative). For example, a reagent test area may be designed to have lines of gray appear in the test area if the test has been activated (e.g., a liquid applied), but is still negative for the medical condition that the test was designed to identify.

In certain examples, reagent test areas may have more than two states. For example, a portion (e.g., a stripe or the like) of a reagent test area may react to “ordinary” liquid and another portion may react to a specific medical condition (e.g., to indicate a urinary tract infection). Thus, a first state may be a not-yet activated state, a second state may be an activated state in which the ordinary portion of the test area is activated (e.g., wetness has been detected), and a third state may correspond to detection of a specific medical condition (e.g., to visually show the presence of UTI or other medical condition) in addition the ordinary portion. The different portions of a given test area may change to different colors—e.g., a stripe in the test area that turns gray may indicate “wetness,” while another stripe that turns red may indicate a positive UTI test.

In certain examples, a test strip may include a dedicated “wetness” reagent test area that is designed to change color upon an application of any “ordinary” liquid (e.g., urine that does not include chemical markers that may be indicative of medical conditions). Thus, the systems described here may be used to detect wetness and provide a notification that, for example, a patient needs a diaper change.

In any event, if the diaper tag determines that the test strip has been activated, image and/or light frequency data or the like (e.g., light wavelength and/or frequency data as described above) of the test strip test areas are acquired in step 728. For example, image data may be acquired and converted into light frequency data. In certain examples, acquisition may be storage of previously acquired optically sensed data. In other words, optically sensed data may be acquired (e.g., sampled) once every 60 seconds (e.g., in step 726) and may only be “stored” for further processing if the diaper tag detects that the test areas have been activated (e.g., if the test areas are wet).

In step 730, the image and/or light frequency data are transmitted to a remote computing system (e.g., using a wireless transmitter). In certain example embodiments, a device tag includes two or more types of sensors, one sensor may be a wetness sensor (e.g., activation of the test areas or perhaps merely electrical connection between conductive areas) and one sensor may be an imaging sensor used to acquire image data regarding the test areas. In certain examples, a frequency-to-color may be used to detect “color” associated with the wetness state of the test strip.

An example system allows authentication that a genuine test strip is in use. The diaper tag will read the data from an embedded passive RFID tag position in the test strip. The example system may perform a check on a remote server against EMR (electronic medical records) so that the particular test in question may be verified as one that was prescribed by a physician. For example, suppose a patient was prescribed to have a wetness ONLY indication during ALL day and ONE test for UTI (urinary tract infection) at 6:00 am. If a nurse applies a normal wetness test at 6:00 am the system may prompt to the nurse to change.

In certain examples, this system may work in conjunction with RFID location tags that are described in related applications—U.S. application Ser. No. 14/267,817 and U.S. Publication No. 2013/0182382, the entire contents of each being hereby incorporated by reference. There may be even further cross-matching with the diaper tag and the personal band worn by a patient so that the system is sure (e.g., by proximity of the diaper tag to the personal band) that the diaper tag (and perhaps a corresponding test strip) is applied to the correct person. This is a further authentication for the quality of data gathered from the diaper (since it is automatically sent without relying upon handwritten notes).

It will be appreciated that certain steps or elements may be removed. For example, the alignment verification process or the check against a particular patient's medical records may be skipped.

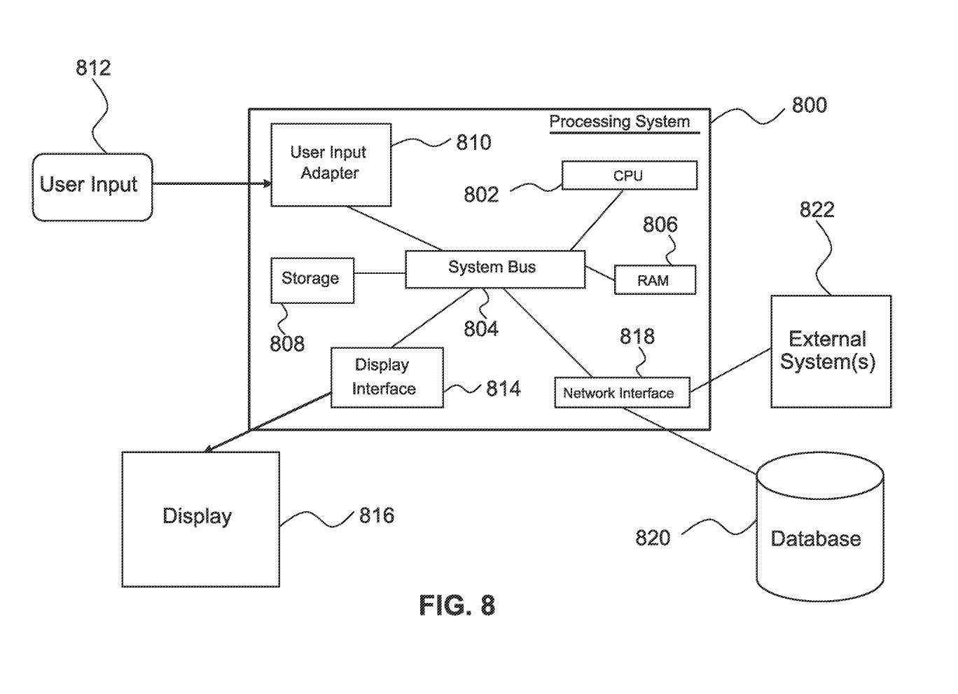

FIG. 8 is a block diagram of an exemplary computing system according to certain example embodiments. A processing system 800 includes a central processing unit or CPU 802, a system bus 804 that communicates with RAM 806, and storage 808. The storage 808 can be magnetic, flash based, solid state, or other storage technology. The system bus 804 may also communicate with a user input adapter 810 (e.g., PS/2, USB interface, or the like) that allows users to input commands to the processing system via a user input device 812 (e.g., a keyboard, mouse, touch panel, or the like). The results of the processing may be displayed to a user on a display 816 via a display interface 814 (e.g., a video card or the like).

The processing system 800 may also include a network interface 818 that may facilitate wired (e.g., Ethernet) or wireless communication (Wi-Fi/802.11x protocols, cellular technology, and the like) with external systems 822 or databases 820. External systems 822 may include other processing systems, systems that provide third party services, etc.

As described herein, external systems 822 may include example diaper tags (and the associated components therein) or a central server system. Additionally, the processing system 800 may implement functionality as a central server system (e.g., where the external system is a diaper tag). Further, a processor included in an example diaper tag may include some (or all) of the components of processing system 800. For example, the network interface may correspond to a transceiver of the diaper tag.

External systems 822 may include other types of computing systems such as, for example, network attached storage (NAS) that holds large amounts of data (e.g., thousands or millions of electronic documents/records). Such external systems, along with the internal storage and memory, may form a storage system for storing and maintaining information on the test results of one or more patients (e.g., thousands of patients). Such a system many communicate with certain users and respective computing resources (e.g., a client system, terminal, etc) to provide test results and associated analysis for review and consideration. The database 820 may include relational, object orientated, or other types of databases for storing information (e.g., such as test results obtained from diaper tags).

The processes, techniques, and the like, described herein are, at least in part, implemented by a computing system. Such implementations include configurations (executable computer program code structures—e.g., sometimes referred to as software) of processing systems to carry out certain aspects of example embodiments. It will be appreciated that implementing such configurations on general purpose hardware (e.g., a generic SoC) may then effectively create special purpose hardware that implements the techniques described herein.

Certain examples herein are described in terms of sequences of actions that can be performed by, for example, elements of a programmable or programmed computer system. It will be recognized that various actions also could be performed by specialized circuits (e.g., discrete logic gates interconnected to perform a specialized function or application-specific integrated circuits—ASIC), by program instructions executed by one or more processors, or by a combination of both.

Moreover, portions of the example embodiments can also be considered as embodied entirely within any form of non-transitory computer-readable storage medium (e.g., RAM, ROM, so-called hard drives, portable media—DVDs, etc) having stored therein an appropriate set of computer readable or executable instructions for use by or in connection with an instruction-execution system, apparatus, or device, such as a computer-based system, processor-containing system, or other system that can fetch instructions from a medium and execute the instructions.

It will be appreciated that while certain examples provided herein are given with respect to a diaper that other garments—e.g., pants, shorts, etc. may also be used.

While certain embodiments herein include a camera, imager, or other optical sensor, other types of sensors may be used to acquire information from a test strip. For example, a diaper tag may include ultrasonic sensors and/or capacitive sensors to acquire information from a test strip—or sensing an electrical circuit between conductive areas in or on absorbent material that is used in a test strip.

Thus, the invention may be embodied in many different forms, not all of which are described above. It will be appreciated that the techniques described herein may be applied to a variety of different contexts. For example, while some examples herein may be in a hospital or formal medical setting, the techniques and embodiments herein also may be applied in a home environment.

While the technology herein has been described in connection with what is presently considered to be preferred embodiment, it is to be understood that the invention is not limited to the disclosed embodiment, but on the contrary, is intended to cover modifications and equivalent arrangements as now will be apparent to those skilled in the art and as included within the spirit and scope of the claims.

What is claimed is:

1. A test strip comprising:
a liquid absorbing material extending along the test strip, the liquid absorbing material including a first area to receive liquid from an external source and a second area separately located away from the first area;
at least one reagent test portion disposed at the second area of the liquid absorbing material and configured to change optical state when exposed to liquid having at least one pre-determined attribute; and
an outer shell made of a substantially liquid impermeable material, the outer shell encompassing the liquid absorbing material and the at least one reagent test portion, the outer shell having at least one aperture disposed at the first area allowing liquid to enter into the outer shell,
wherein a portion of the outer shell located proximate the second area allows acquisition of optical signals from the at least one reagent test portion.

2. The test strip of claim 1, wherein the at least one aperture comprises a plurality of apertures formed through the substantially liquid impermeable material of the outer shell proximate the first area.

3. The test strip of claim 1, wherein the at least one aperture comprises a one-way liquid permeable membrane allowing liquid to flow into the outer shell proximate the first area.

4. The test strip of claim 1, further comprising:
at least one electrically conductive path extending along a path on an outer surface of the outer shell.

5. The test strip of claim 4, wherein the at least one electrically conductive path includes a pair of parallel conductive strips that extend at least partly along opposing sides of the outer shell.

6. The test strip of claim 1, wherein the outer shell includes a pair of attachment apertures that extend through one surface of the outer shell, through the liquid absorbing material, and through an opposing surface of the outer shell.

7. The test strip of claim 1, wherein the outer shell is made of nylon with a window portion at the second area being substantially clear, translucent, or transparent and the remainder of the substantially liquid impermeable material of the outer shell being substantially opaque.

8. The test strip of claim 1, further comprising:
an adhesive strip affixed to an external surface of the outer shell, the adhesive strip including an adhesive surface disposed to secure the test strip to an external surface of a clothing article.

9. The test strip of claim 1, further comprising:
a comfort layer disposed over the outer-shell at the first area for contact with skin of a person.

10. The test strip of claim 1, wherein a thin layer of liquid absorbing material is interposed between the at least one reagent test portion and the outer shell.

11. A liquid analysis system for testing liquid from a person, the system comprising:
a test strip configured for disposition on an inner surface of a garment to be worn by a person, the test strip including at least one external electrically conductive path and at least one internal test portion that changes optical state if contacted by a liquid having a predetermined attribute;
a housing configured for removable attachment to the test strip and to the garment, the housing including at least one surface having a plurality of electrically conductive contact areas that are configured to electrically communicate with the at least one electrically conductive path when attached to the test strip;
at least one optical sensor carried by the housing and configured to view the at least one test portion when the housing is attached to the test strip and to the garment; and
at least one electrical processing circuit carried within the housing and electrically coupled to the at least one optical sensor and to the electrically conductive contact areas, the electrical processing circuit being configured to perform, or to assist in performing, at least in part, an alignment verification process to determine if the at least one test portion is properly aligned with respect to the housing by determining whether or not the plurality of electrically conductive contact areas effect predetermined electrical communications with the at least one electrically conductive path.

12. The liquid analysis system of claim 11, wherein the at least one processing circuit is further configured to, at least in part, control and permit the housing to be secured to the garment and/or test strip only if the at least one test portion is determined to be properly aligned with respect to the housing.

13. The liquid analysis system of claim 11, wherein the at least one processing circuit is further configured to trigger notification to a user if the housing is determined not to be properly aligned with respect to the at least one test portion as a result of the alignment verification process.

14. The liquid analysis system of claim 11, wherein the at least one processing circuit is further configured to, at least in part, controllably prevent the housing from being secured to the garment based on a result of the alignment verification process.

15. A test strip comprising:
an outer shell enclosing an inner area and made of a substantially liquid impermeable material, the outer shell having a window portion configured to pass optical signals between at least a portion of the inner area and an exterior of the outer shell, the outer shell having at least one aperture at a liquid transmission portion that is separately located away from the window portion, the at least one aperture being configured to allow liquid transmission through the substantially liquid impermeable material of the outer shell and into the inner area;
a liquid absorbing material disposed in the inner area of the outer shell and extending from the liquid transmission portion to the window portion;
at least one reagent test area disposed within the inner area of the outer shell proximate the window portion, the at least one reagent test area being configured to change optical state based on application of liquid thereto; and
at least one electrically conductive path in a predetermined pattern on an external surface of the outer shell.

16. The test strip of claim 15, wherein the at least one electrically conductive path is disposed on an external surface of the outer shell as a pair of conductive strips on opposing sides of the window portion.

17. The test strip of claim 15, wherein the liquid absorbing material substantially encompasses the at least one reagent test area.

18. The test strip of claim 17, wherein a thin layer of liquid absorbing material is disposed between the at least one reagent test area and the window portion of the outer shell.

19. The test strip of claim 15, wherein the outer shell has a pair of attachment apertures extending from a first side of the outer shell to and through an opposing side of the outer shell.

20. The test strip of claim 15, further comprising an external tissue layer disposed outside the outer shell over the liquid transmission area for contact with skin of a user.
